(19) United States
(12) Patent Application Publication
(10) Pub. No.: US 2011/0167674 A1
(43) Pub. Date: Jul. 14, 2011

(54) Rearfoot Post for Orthotics

(76) Inventor: Paul Stuart Langer, Fort Salonga, NY (US)

(21) Appl. No.: 12/986,289

(22) Filed: Jan. 7, 2011

Related U.S. Application Data
(60) Provisional application No. 61/293,856, filed on Jan. 11, 2010.

(51) Int. Cl. A43B 21/00; A61F 5/14

(52) U.S. Cl. 36/82; 36/144

(57) ABSTRACT

A rearfoot post comprises a stop segment and an elastic segment operatively coupled along an axis of rotation. The stop segment is fabricated from a firm or rigid material. The elastic segment compresses and expands in response to foot motion. The elastic segment can include an elastomer or a spring. In some embodiments, the stop segment and the elastic segment are operatively coupled by a hinge, and the axis of rotation coincides with the axis of the hinge. Depending on service applications, a plate can be attached to the bottom of the stop segment and to the bottom of the elastic segment. Embodiments of the rearfoot post also include a heel cup. A heel cup with a flat bottom is advantageous for controlling stability of the foot and reducing shock on the heel.

This application claims the benefit of U.S. Provisional Application No. 61/293,856 filed Jan. 11, 2010, which is incorporated herein by reference.

### BACKGROUND OF THE INVENTION

The present invention relates generally to orthotics, and more particularly to a rearfoot post for orthotics.

Functional foot orthotics (“orthotics”) are worn in a shoe during standing, walking, or running to influence the orientation of the bones of a human foot with respect to each other, to influence the orientation of the bones of the foot with respect to the bones of the ankle or leg, and to influence the direction and force of motion of the foot or parts of the foot. More than one of these influences can be applied to the whole foot or parts of the foot at various times during a sequence of motions that make up walking or running; this sequence is referred to as “the gait cycle”. More than one influence can be applied simultaneously to any particular part of the foot during the gait cycle. Different influences can be applied to the whole foot or particular parts of the foot at various points in the gait cycle.

Many orthotics employ a feature known as a rearfoot post to influence the motion of the subtalar joint (a joint made up of the talus and the calcaneus bones) known as subtalar joint pronation (“subtalar pronation”). Many orthotics employ one or more features to reduce the effect on the human body of the force of the moving body as the shoe makes contact with the ground (“shock”) during walking, running, or jumping.

Orthotics often include a component (referred to as the “shell”) formed from a material that has been molded or otherwise shaped to approximately conform to part or all of the plantar surface of the foot. The earliest orthotics had rigid shells with rigid rearfoot posts applied to the proximal portion of the underside of the shell. The bottom-most surface of the rearfoot post was shaped into two intersecting planes or facets. When resting on a hard, flat surface, the orthotic would rock or rotate around an axis that lies along the intersection of the two planes. The angular relationship between the two planes could be used to limit the amount of rotation of the orthotic. The axis of rotation could be varied by changing the relative position of the two intersecting planes. It was assumed that, when the orthotic was worn inside a shoe, the rotation of the foot along the same axis as the orthotic could be controlled and, if the axis of rotation was parallel to the axis of rotation of subtalar pronation, the amount of subtalar pronation could be controlled.

Later orthotics had flexible shells and compressible rearfoot posts applied to the proximal portion of the underside of the shell. The bottom-most surface of the rearfoot post was a single plane fixed at an angle relative to the bottom-most surface of the shell. The angle of this plane was such that the rearfoot post was thicker on the medial side and thinner on the lateral side of the orthotic. This post was in essence a wedge worn under the heel of the foot and held the rearfoot in an inverted position from the beginning of the gait cycle until the center of mass of the body passed forward onto the distal portion of the orthotic.

In practice, neither of the methods described above achieved the goal of limiting subtalar pronation in most shoes. The earlier rigid post created an indentation in the comparatively soft material of the shoe. The orthotic sank into the indentation and became immobile. The later version had the same problem, as well as additional complications. Upon first wearing of the orthotic, the wedging effect would move the axis of rotation to the exterior of the shoe, thereby increasing the length of the lever arm of the frontal plane component of rearfoot pronation. Since frontal plane rotation is the dominant component of rearfoot pronation, lengthening the lever arm of pronation reduces the ability of the rearfoot post to control rearfoot pronation. As the orthotic was worn, the compressible material of the rearfoot post would permanently compress and deform; consequently, the flat plane of the rearfoot post would become curved. The resulting curved shape created an indeterminate axis of rotation and an indeterminate amount of rotation.

Some variations of the compressible rearfoot post had lower density material on the lateral side than on the medial side. The softer material on the lateral side was intended to absorb shock. In practice, the softer compressible material deformed more quickly and became more curved, resulting in a less determinate shape.

### BRIEF SUMMARY OF THE INVENTION

A rearfoot post comprises a stop segment and an elastic segment operatively coupled along an axis of rotation. The stop segment is fabricated from a firm or rigid material. The elastic segment compresses and expands in response to foot motion. In some embodiments, the elastic segment includes an elastomer. In other embodiments, the elastic segment includes a spring. In some embodiments, the stop segment and the elastic segment are operatively coupled by a hinge, and the axis of rotation coincides with the axis of the hinge. In other embodiments, no hinge is used, and the axis of rotation is defined by the boundary between the stop segment and the elastic segment. Depending on service applications, embodiments include a plate attached to the bottom of the stop segment, a plate attached to the bottom of the elastic segment, or a plate attached to the bottom of the stop segment and a plate attached to the bottom of the elastic segment. Embodiments of the rearfoot post also include a heel cup. A heel cup with a flat bottom is advantageous for controlling stability of the foot and reducing shock on the heel.

These and other advantages of the invention will be apparent to those of ordinary skill in the art by reference to the following detailed description and the accompanying drawings.

### BRIEF DESCRIPTION OF THE DRAWINGS

FIG. 1 shows a prior-art orthotic;

FIG. 2 shows an orthotic, according to an embodiment of the invention;

FIG. 3 shows a prior-art heel cup;

FIG. 4 shows a heel cup, according to an embodiment of the invention;

FIG. 5 shows the distribution of forces on the heel;

FIG. 6A-FIG. 6C show details of a heel cup, according to an embodiment of the invention;

FIG. 7A-FIG. 7I show a rearfoot post, according to an embodiment of the invention;

FIG. 8A-FIG. 8I show a rearfoot post with an elastic segment including an elastomer, according to an embodiment of the invention;

FIG. 9 shows a rearfoot post including a heel cup, according to an embodiment of the invention;

FIG. 10 shows a reference Cartesian coordinate system;

FIG. 11A-FIG. 11K show a rearfoot post with an elastic segment including a spring, according to an embodiment of the invention;

FIG. 12A and FIG. 12B show a rearfoot post with an elastic segment including a spring, according to an embodiment of the invention; and

FIG. 13A-FIG. 13D show the reference geometry for axes of rotation.

### DETAILED DESCRIPTION

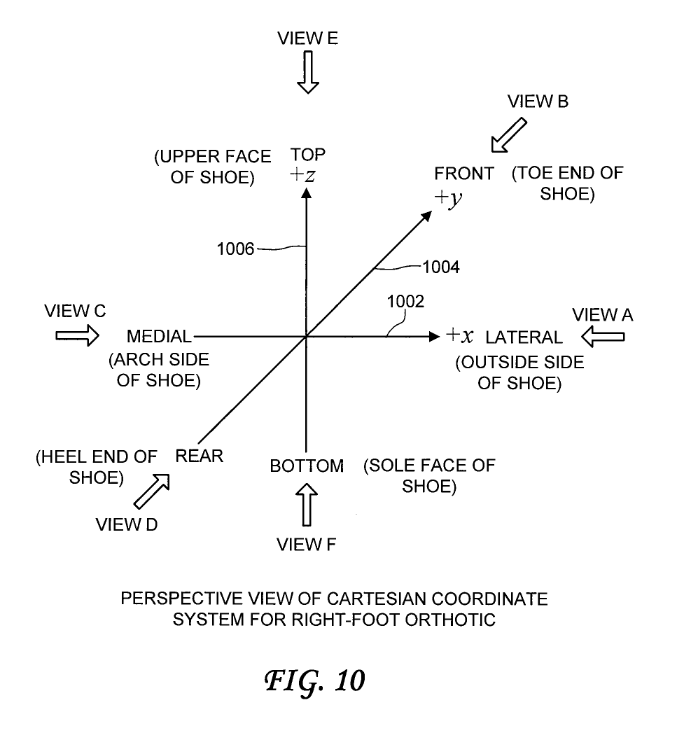

In the design of orthotics, the geometries are generally complex, and reference points and axes relative to the human body are often used. Herein, the Cartesian coordinate system shown in FIG. 10 (perspective view) is used in descriptions of embodiments of rearfoot posts. A right-foot orthotic is used in the examples. Corresponding geometries apply for a left-foot orthotic. The Cartesian coordinate system is defined by the x-axis 1002, the y-axis 1004, and the z-axis 1006. The +x direction points towards the lateral side of the shoe (the outside of the shoe away from the midline of the body). The −x direction points towards the medial side of the shoe (the arch side of the shoe towards the midline of the body). The +y direction points towards the front end of the shoe (the toe end of the shoe). The −y direction points towards the rear end of the shoe (the heel end of the shoe). The +z direction points towards the top face of the shoe (the upper face of the shoe). The −z direction points towards the bottom face of the shoe (the sole face of the shoe).

View A is sighted along the −x direction. View B is sighted along the −y direction. View C is sighted along the +x direction. View D is sighted along the +y direction. View E is sighted along the −z direction. View F is sighted along the +z direction.

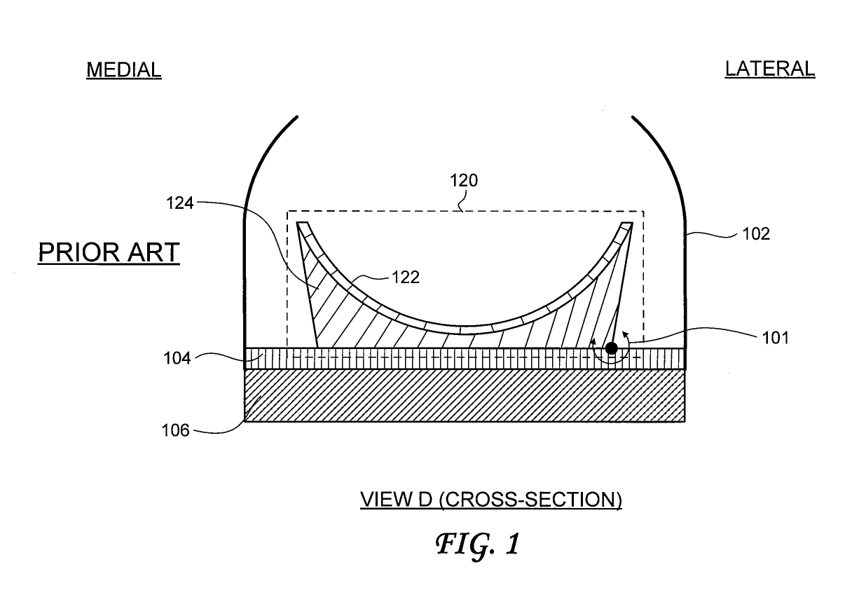

FIG. 1 (View D cross-section) shows the rotational geometry of a prior-art rearfoot post fitted inside a shoe. The parts of the shoe shown are the shoe uppers 102, the insole 104, and the heel 106. The orthotic 120 includes a shell 122 and a rearfoot post 124. The axis of rotation of the rearfoot post 124 is denoted the axis of rotation 101. Note that the entire rearfoot post 124 rotates about the axis of rotation 101.

According to an embodiment of the invention, a rearfoot post utilizes the action of an elastic segment on the lateral side combined with a stop segment on the medial side. The elastic segment alternately compresses and expands to create motion along a predefined (user-specified) axis of rotation. Herein, a user refers to a person specifying the design of the orthotic. For custom orthotics, the user will typically be a podiatrist treating a patient. Embodiments of the invention can also be used for non-custom orthotics (such as mass-market orthotics) with an assortment of axes of rotation specified by a podiatrist or others skilled in the art of orthotic design. The axis of rotation can be varied by varying the position and orientation of the line along which the elastic segment and the stop segment intersect. The motion of the rearfoot post is within the rearfoot post itself. If the rearfoot post becomes embedded in the soft material of the shoe (such as the insole of the shoe), it will not become immobilized.

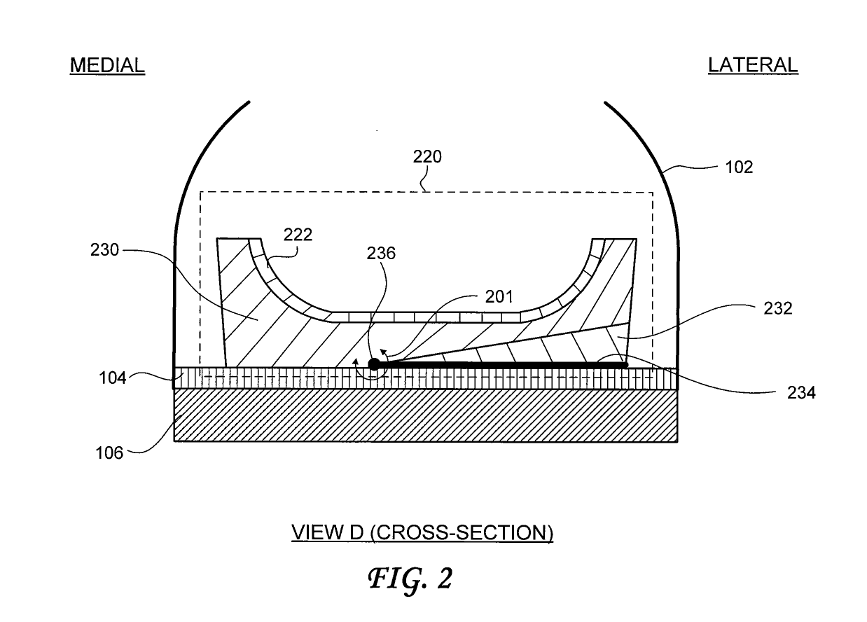

FIG. 2 (View D cross-section) shows the rotational geometry of a rearfoot post according to an embodiment of the invention. The orthotic 220 includes a shell 222 and a rearfoot post. The rearfoot post includes a stop segment 230 and an elastic segment including an elastomer 232 and a movable plate 234. The movable plate 234 pivots about a hinge 236. The axis of rotation 201 coincides with the axis of hinge 236. The elastomer 232 compresses and expands during foot motion. More detailed descriptions of embodiments of the invention are provided below.

Since the axis of rotation of the rearfoot post is internal to the shoe and medial to the heel of the foot, the length of the lever arm of the frontal plane component of rearfoot pronation is decreased. As discussed previously, frontal plane rotation is the dominant component of rearfoot pronation; consequently, shortening the lever arm of pronation increases the ability of the rearfoot post to control rearfoot pronation. The elastic segment on the lateral side of the rearfoot post also absorbs shock and reduces its effect on the body. The absorption of shock by the lateral side of the rearfoot post reduces the force acting on the medial side of the rearfoot post, thereby reducing the wear on the medial side of the rearfoot post. In addition, the elastic segment on the lateral side of the rearfoot post will not permanently deform. These factors prevent the bottom-most surface of the rearfoot post from becoming deformed from the desired shape over time.

Embodiments of the rearfoot post described herein can be incorporated into any typical foot orthotic worn inside a shoe, ranging from a heel cup only orthotic to one that partially fills the bottom of the interior of a shoe to one that fills the entire bottom of the interior of a shoe. Embodiments can also be incorporated into any leg brace or ankle-foot orthotic. In addition to embodiments that can be inserted into and removed from a shoe, other embodiments can be integrated into a shoe (for example, prescription footwear).

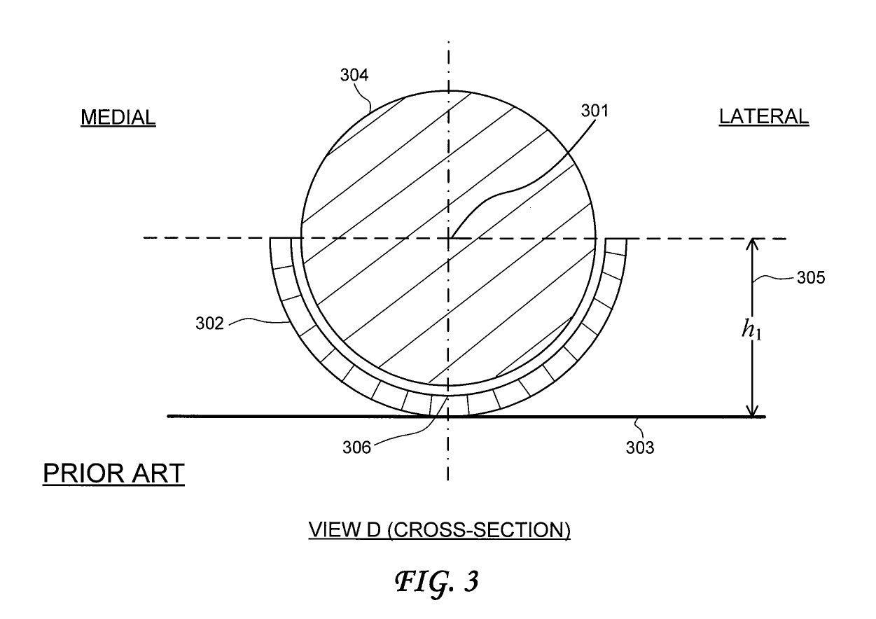

FIG. 3 (View D cross-section) shows a schematic of a prior-art heel cup. The rearfoot post includes a heel cup 302 with a curved bottom 306. Heel 304 is fully constrained by heel cup 302. For illustration purposes only, heel 304 and heel cup 302 are shown with a slight separation. In actual practice, heel 304 is in contact with heel cup 302. Anatomical reference point 301 is an anatomical reference point within the heel 304. The height h₁ 305 specifies the height of the anatomical reference point 301 above a reference plane 303. Heel cup 302 can be designed with or without a shell.

FIG. 4 (View D cross-section) shows a schematic of a heel cup according to an embodiment of the invention. The heel portion of the rearfoot post has a flat bottom in the center and is curved on the medial and lateral sides. The geometry and dimensions can be user-specified for a specific patient's foot. Heel 304 is supported by a heel cup 402 with a flat bottom 406. The height h₂ 405 specifies the height of the anatomical reference point 301 above the reference plane 303. The height h₂ 405 in FIG. 4 is less than the corresponding height h₁ 305 in FIG. 3. Some embodiments of heel cup 402 have a shell; some embodiments of a heel cup 402 do not have a shell.

The flat bottom of the heel cup 402 creates a more stable base of support for the calcaneus. The lower height h₂ 405 adds to the stability of the heel. The flat bottom and curved sides of the heel cup 402 allow for the fluid motion of the fat pad under the heel of the foot as the shoe makes contact with the ground. In FIG. 4, the original fat pad of heel 304 is denoted original fat pad 408A. As the heel is inserted into the heel cup 402, the fat pad expands to expanded fat pad 408B. The flat bottom and curved sides of the heel cup 402 allow the foot to sit lower in the orthotic, creating a better fit between the foot, the orthotic, and the shoe. Note that a heel cup with a flat bottom is also advantageous for prior-art rearfoot posts without separate stop segments and elastic segments (such as shown in FIG. 1).

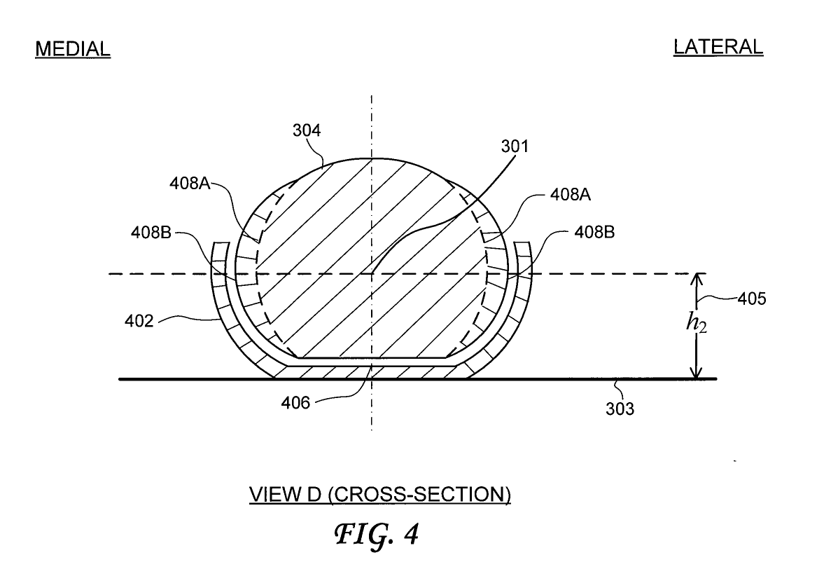

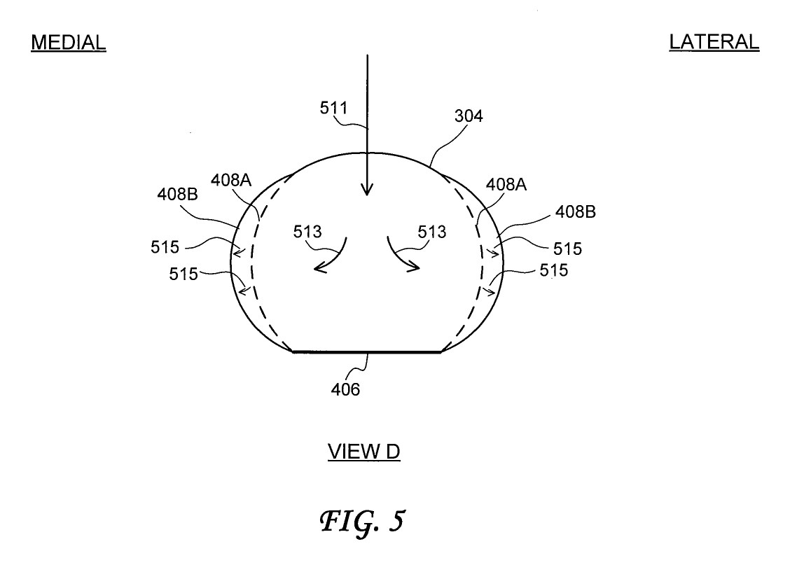

A further advantage of the flat bottom accrues because the fluid motion of the fat pad also dissipates shock and reduces its effect on the body. Refer to FIG. 5 (View D). Heel 304 rests on the flat bottom 406. The initial shock (represented by force vector 511) is dissipated by the fat pad; force vectors 513 represent the forces at the original fat pad 408A. If the fat pad is allowed to expand to expanded fat pad 408B, the force is further dissipated; force vectors 515 represent the forces at expanded fat pad 408B. In the prior-art heel cup 302 shown in FIG. 3, however, the fat pad is constrained from expanding.

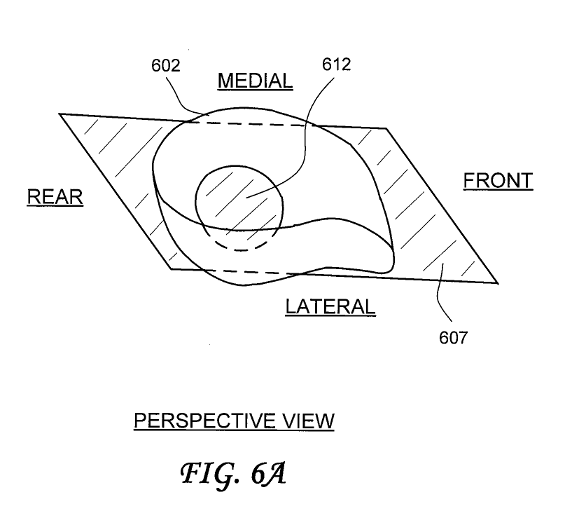

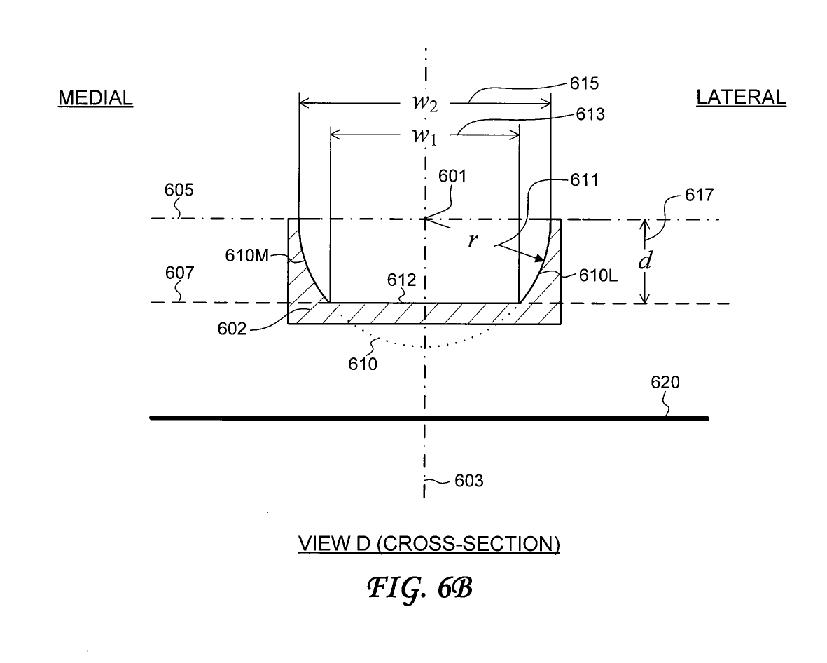

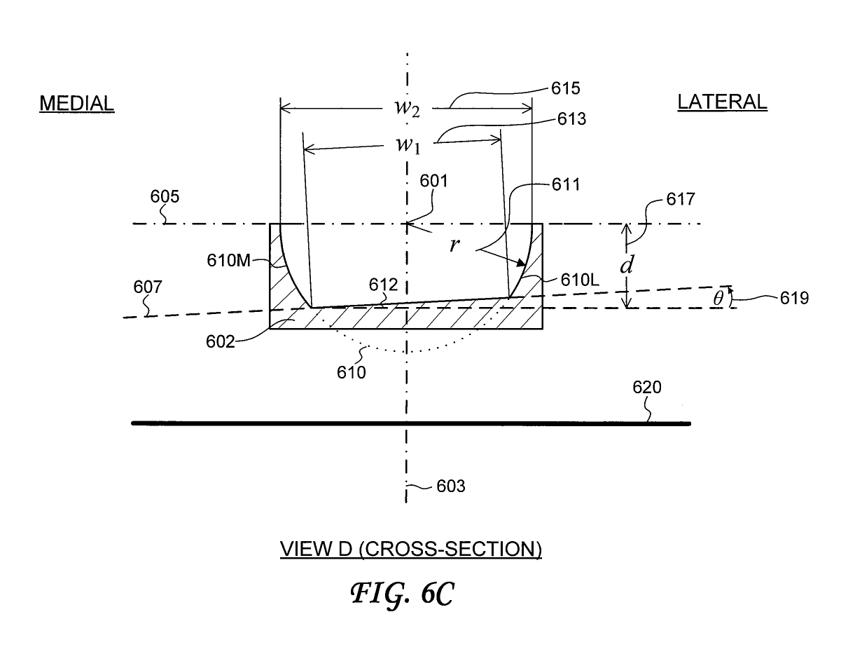

Refer to the perspective view shown in FIG. 6A. In an embodiment of the invention, the rearfoot post is attached to a rigid or flexible shell made of material that has been molded or otherwise shaped to partially conform to the plantar, medial, and lateral surfaces of a human foot in the area of the heel to form heel cup 602. The flat bottom 612 is specified by the intersection of a reference plane 607 with the shell.

Refer to FIG. 6B (View D cross-section). The reference axis 603 is the midline of the heel. The reference axis 605 is perpendicular to the reference axis 603 and passes across the top of heel cup 602. Reference axis 603 and reference axis 605 intersect at center point 601. The ground is represented by ground plane 620. Reference axis 605 is parallel to ground plane 620.

Medial side 610M and lateral side 610L are arcs of a reference circle 610 with a center at center point 601 and a radius r 611. The flat bottom 612 is formed by the intersection of reference circle 610 with reference plane 607 located at a depth d 617 below reference axis 605. In the embodiment shown in FIG. 6B, reference plane 607 is parallel to ground plane 620. The width of flat bottom 612 is width w₁ 613. The full width of heel cup 602 is width w₂ 615, where w₂=2r. In an embodiment, w₁≧˜w₂/3.

Refer to FIG. 6C (View D cross-section). In the embodiment shown, the reference plane 607 intersects reference circle 610 at an angle θ 619 with respect to the ground plane 620. The angle θ is measured about an axis lying in one or more body planes. The higher the angle θ, the more leverage the device will have to control or induce motion in the foot. In an embodiment, w₁≧˜w₂/3.

In the embodiments shown in FIG. 6A-FIG. 6C, the heel cup is formed from a shell, and the medial side and lateral side have contours that are arcs of a circle. In other embodiments, the heel cup is a portion of a rearfoot post without a shell. In other embodiments, the medial side and lateral side have user-specified curved contours that are not arcs of a circle. Note that the contour of the medial side can be different from the contour of the lateral side.

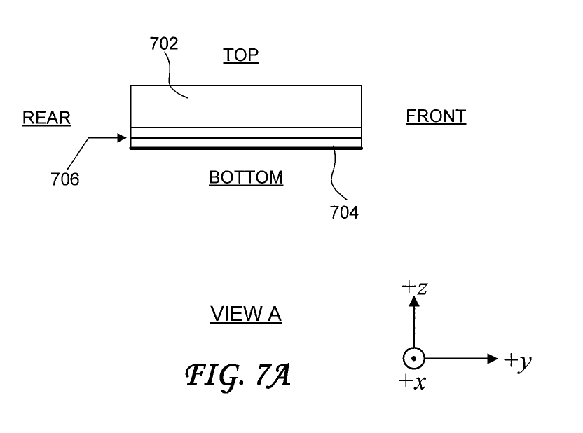

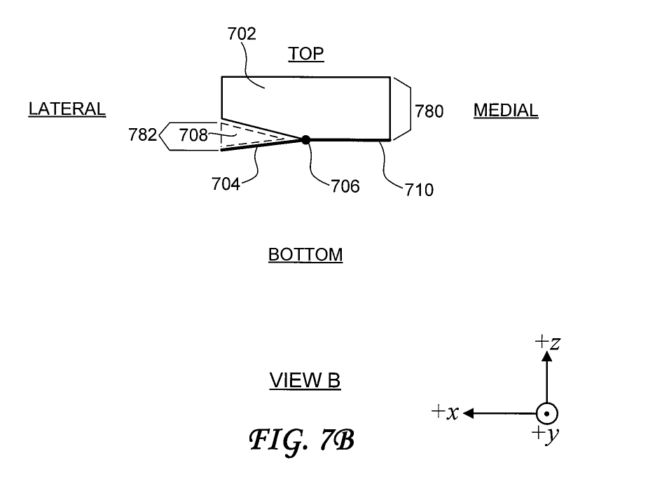

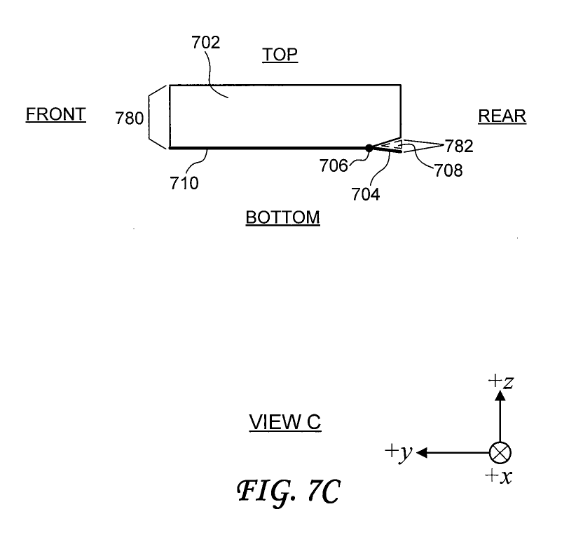

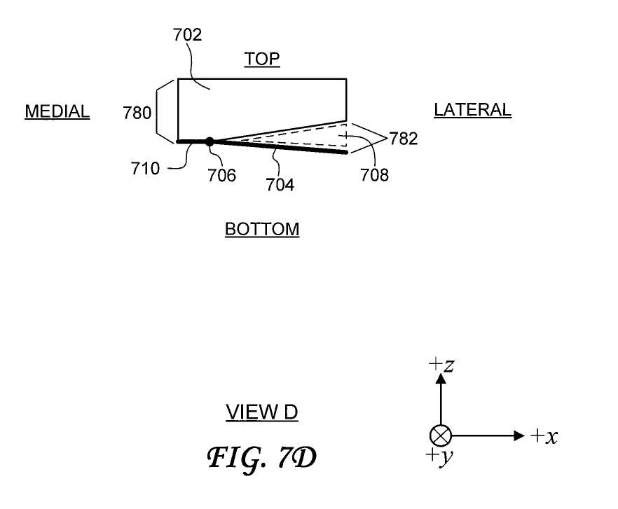

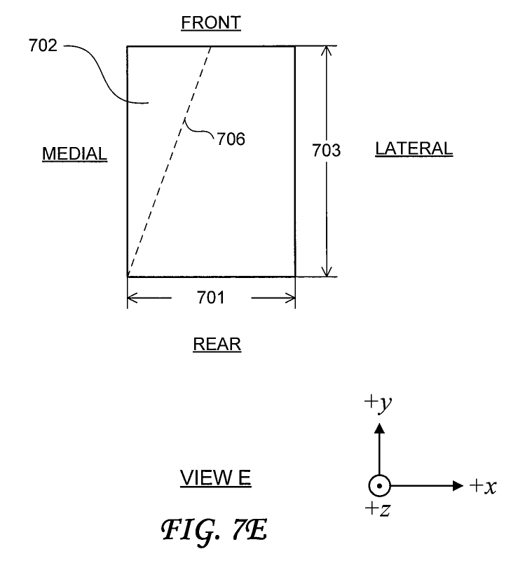

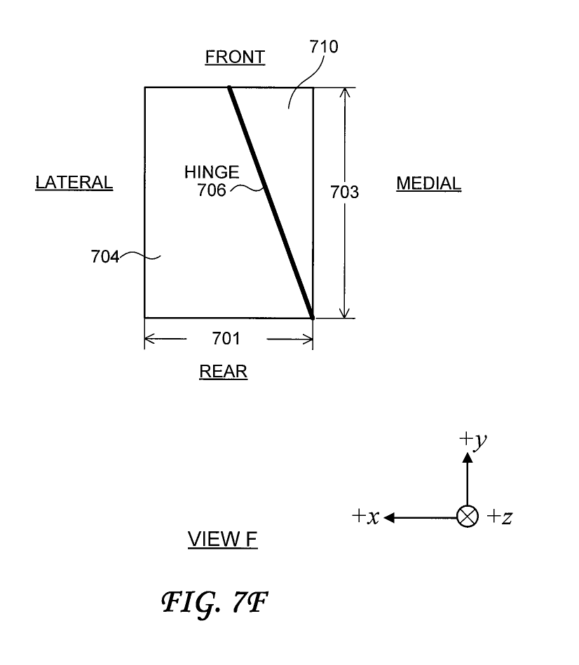

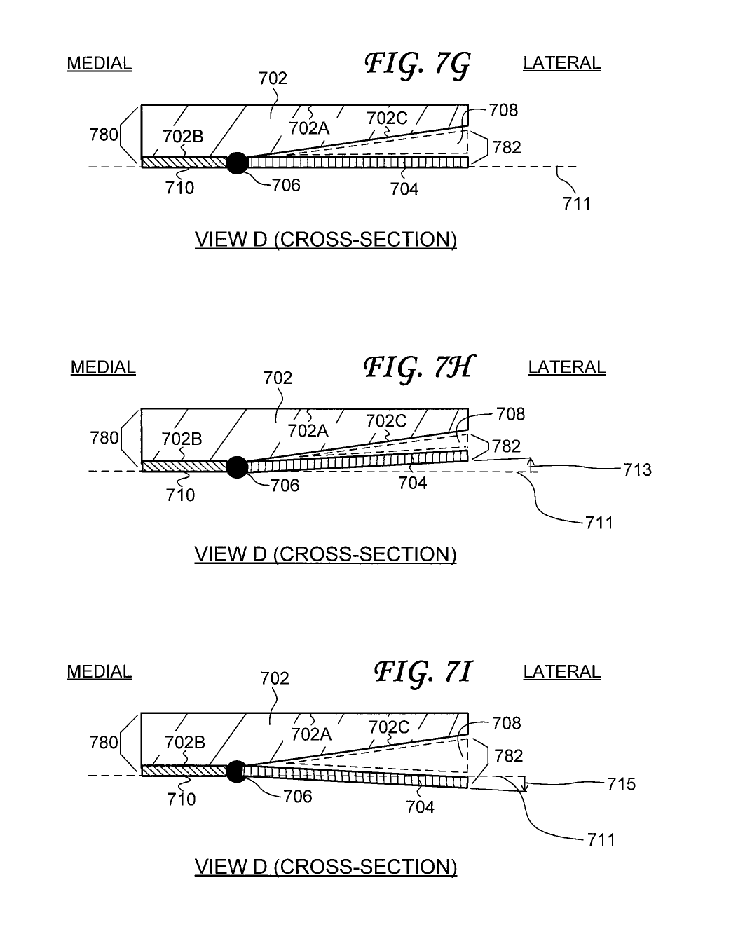

The top of a rearfoot post can be flat instead of cup-shaped; that is, the curved sides on the medial and lateral sides are absent. FIG. 7A-FIG. 7F show View A-View F, respectively, of a rearfoot post according to an embodiment of the invention. Note that this embodiment has no shell. Other embodiments have a shell. Refer to FIG. 7D (View D). The rearfoot post includes a stop segment 780 and an elastic segment 782. The stop segment 780 includes a platform 702 fabricated from a firm or rigid material (in an embodiment, a material with a hardness of approximately 35 or greater Shore A durometer) and a fixed plate 710 attached to the bottom of platform 702 on the medial side. The elastic segment 782 includes a compression-expansion zone 708 (described in more detail below) and a movable plate 704 attached to the bottom of the compression-expansion zone 708. The movable plate 704 is operatively coupled to the fixed plate 710 by a hinge 706. During a gait motion, the elastic segment 782 alternately compresses and expands. Note that fixed plate 710 is fixed with respect to platform 702, and movable plate 704 is movable with respect to platform 702.

In some embodiments, the hinge is a standalone unit, and the fixed plate and the movable plate are attached to it. In other embodiments, a portion of the hinge is integrated into the fixed plate and a portion of the hinge is integrated into the movable plate. The two portions of the hinge interlock, and the movable plate can rotate with respect to the fixed plate.

Refer to FIG. 7E (View E) and FIG. 7F (View F). To simplify the drawings, in the embodiment shown, the top and bottom surfaces of the rearfoot post have a rectangular geometry with a width 701 and a length 703. In general, the top and bottom surfaces can have a user-specified combination of linear and curvilinear geometry to conform to the foot and the shoe. In general, the top surface can have a different geometry from the bottom surface.

FIG. 7A-FIG. 7C show additional views for clarity. The features shown in FIG. 7D-FIG. 7F are denoted by the same reference numbers in FIG. 7A-FIG. 7C.

FIG. 7G-FIG. 7I (View D cross-section) show three different initial orientations of the movable plate 704 when the elastic segment 782 is in the relaxed (uncompressed) state. In the figures, reference plane 711 is parallel to the top surface of the rearfoot post. In FIG. 7G, the movable plate 704 is parallel to the reference plane 711 (offset angle is zero). In FIG. 7H, the movable plate 704 is tilted by the offset angle 713 above the reference plane 711. In FIG. 7I, the movable plate 704 is tilted by the offset angle 715 below the reference plane 711.

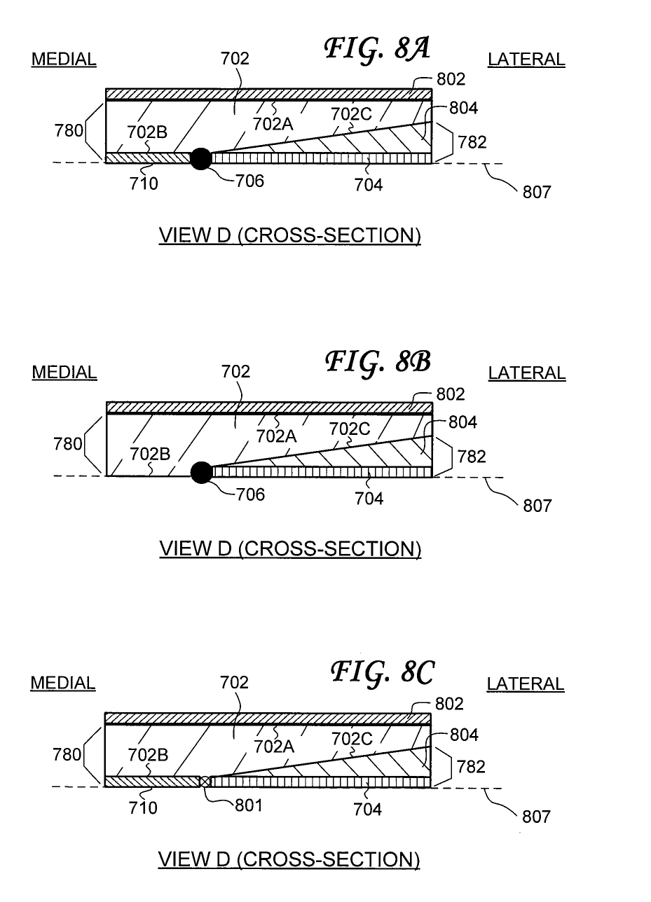

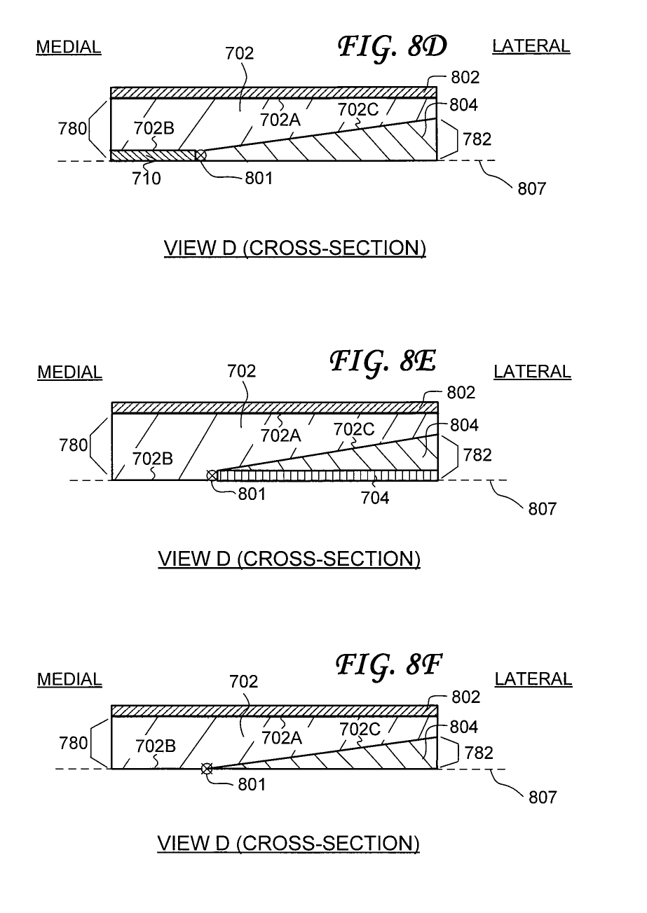

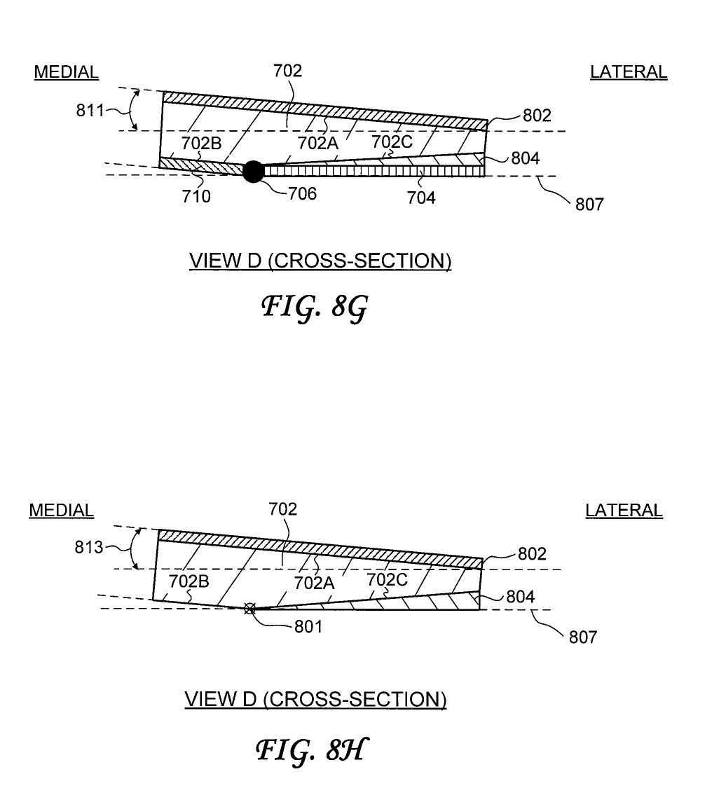

FIG. 8A-FIG. 8F (View D cross-section) show configurations of rearfoot posts according to various embodiments of the invention in which the elastic segment includes an elastomer. Note that these embodiments have a shell. Other embodiments have no shell.

In FIG. 8A, the platform 702 has a top surface 702A, a bottom surface 702B, and an inclined surface 702C. A shell 802 is attached to the top surface 702A. The fixed plate 710 is attached to the bottom surface 702B. Stop segment 780 includes platform 702 and fixed plate 710. The compression-expansion zone 708 (see FIG. 7G) is filled with an elastomer 804 disposed between the inclined surface 702C and the top surface of movable plate 704. In some embodiments, fixed plate 710 and movable plate 704 are fabricated from the same material; in other embodiments, fixed plate 710 and movable plate 704 are fabricated from different materials. Elastic segment 782 includes elastomer 804 and movable plate 704. In this embodiment, elastomer 804 has a wedge shape. The shell 802, fixed plate 710, and movable plate 704 are initially parallel to a reference plane 807 when the elastomer 804 is in an uncompressed state. FIG. 8G (View D cross-section) shows the rearfoot post when the elastomer 804 is in the compressed state. The shell 802 and fixed plate 710 are tilted at an angle 811 about the axis of hinge 706 with respect to the reference plane 807.

The elastic properties of an elastomer can be characterized by various parameters, such as Young's modulus, hardness, and resilience. In general, the measured parameters are dependent on specific measurement instruments and measurement conditions (including temperature and measurement time). The parameter known as rebound resilience is useful for characterizing the elastic properties of elastomers for orthotic applications. In one example, elastomer 804 is a urethane foam with an average rebound resilience of approximately 12-25%, as measured with a vertical ball rebound tester.

The embodiment shown in FIG. 8B is similar to that shown in FIG. 8A, except that the fixed plate is absent. The bottom surface 702B then rests on the insole of the shoe. In this instance, the stop segment 780 includes only platform 702. The movable plate 704 is operative coupled to the platform 702 by the hinge 706.

The embodiment shown in FIG. 8C is similar to that shown in FIG. 8A, except that the hinge 706 is absent. The movable plate 704 flexes about an axis of rotation (AOR) 801. In one embodiment, fixed plate 710 and movable plate 704 are formed from a single sheet of material, and the axis of rotation 801 is defined by the intersection of stop segment 780 and elastic segment 782 on the bottom surface. In a second embodiment, fixed plate 710 and movable plate 704 are formed from a single sheet of material, and the axis of rotation 801 is defined by a notch, score line, indentation line, or ridge line along the sheet of material. In a third embodiment, fixed plate 710 and movable plate 704 are formed from two separate sheets of material, and the axis of rotation 801 is defined by the seam between the two separate sheets. The seam can either be a gap (if the separate sheets are not attached) or a line of attachment (if the separate sheets are attached). In some embodiments, fixed plate 710 and movable plate 704 are fabricated from the same material; in other embodiments, fixed plate 710 and movable plate 704 are fabricated from different materials.

The embodiment shown in FIG. 8D is similar to that shown in FIG. 8C, except that the movable plate 704 is absent. The bottom surface of elastomer 804 then rests on the insole of the shoe. In this instance, elastic segment 782 includes only elastomer 804.

The embodiment shown in FIG. 8E is similar to that shown in FIG. 8C, except that the fixed plate 710 is absent. The bottom surface of platform 702 then rests on the insole of the shoe.

The embodiment shown in FIG. 8F is similar to that shown in FIG. 8C, except that both the fixed plate 710 and the movable plate 704 are absent. The bottom surface 702B and the bottom surface of elastomer 804 then rest on the insole of the shoe. FIG. 8H (View D cross-section) shows the rearfoot post when the elastomer 804 is in the compressed state. The shell 802 and the bottom surface 702B are tilted at an angle 813 about the axis of rotation 801 with respect to the reference plane 807.

FIG. 8I (View D cross-section) shows a dimensional schematic of the rearfoot post previously shown in FIG. 8A. The midline of the rearfoot post is represented by the midline 821. The distance between midline 821 and the medial edge of shell 802 is distance 823. The distance between midline 821 and the lateral edge of shell 802 is distance 825. The distance between midline 821 and the axis of hinge 706 is distance 827. The distance between midline 821 and the medial edge of fixed plate 710 is distance 829. The distance between midline 821 and the lateral edge of movable plate 704 is distance 831. The thickness of shell 802 is thickness 833. The thickness of fixed plate 710 is thickness 839. The thickness of movable plate 704 is thickness 843. The thickness of platform 702 on the medial side is thickness 835. The thickness of platform 702 on the lateral side is thickness 837. The thickness of elastomer 804 on the lateral side is thickness 841. All the distances and thicknesses are user-specified design parameters.

In the embodiments shown in FIG. 8A-FIG. 8F, elastomer 804 has the geometry of a wedge with a planar interface between elastomer 804 and inclined surface 702C and a planar interface between elastomer 804 and movable plate 704. In general, the interfaces can be contoured. In the embodiments shown in FIG. 8A-FIG. 8F, elastomer 804 is a solid, homogeneous material. In general, elastomer 804 can have surface and internal structures, such as holes, channels, honeycombs, and corrugations. In general, elastomer 804 can be a heterogeneous material, including composites; for example, the resilience can vary as a function of distance from the midline. In general, elastomer 804 can include more than one section. The sections can be attached or unattached to one another. The sections can be contiguous (touching) or spaced apart. The sections can be made of the same material or of different materials.

In the embodiments shown in FIG. 8A-FIG. 8F, platform 702 is formed from a solid, homogeneous material. In general, platform 702 can have surface and internal structures, such as holes, channels, honeycombs, and corrugations. In general, platform 702 can be a heterogeneous material, including composites; for example, the hardness can vary as a function of distance from the midline. In general, platform 702 can include more than one section. The sections can be attached or unattached to one another. The sections can be contiguous (touching) or spaced apart. The sections can be made of the same material or of different materials.

Materials suitable for platform 702, fixed plate 710, and movable plate 704 include dense polymer foam, wood, plastic, metal, and ceramic. Depending on the application, fixed plates and movable plates are used for control of various service properties such as rigidity, abrasion resistance, and slip resistance. Note that the choice of materials depends on a variety of factors, such as required foot correction, cost, and service life. For example, if the orthotic is intended for temporary use, a high degree of abrasion resistance is not an important design consideration. If the platform 702 has adequate service properties for a particular application, a fixed plate is not needed. Similarly, if the elastomer has adequate service properties for a particular application, the movable plate is not needed.

In the embodiments shown in FIG. 8A-FIG. 8F, the elastic segment includes an elastomer. In other embodiments, a fluid-filled bladder can be used instead of an elastomer. The bladder can be filled with air or another gas, a liquid, or a gel.

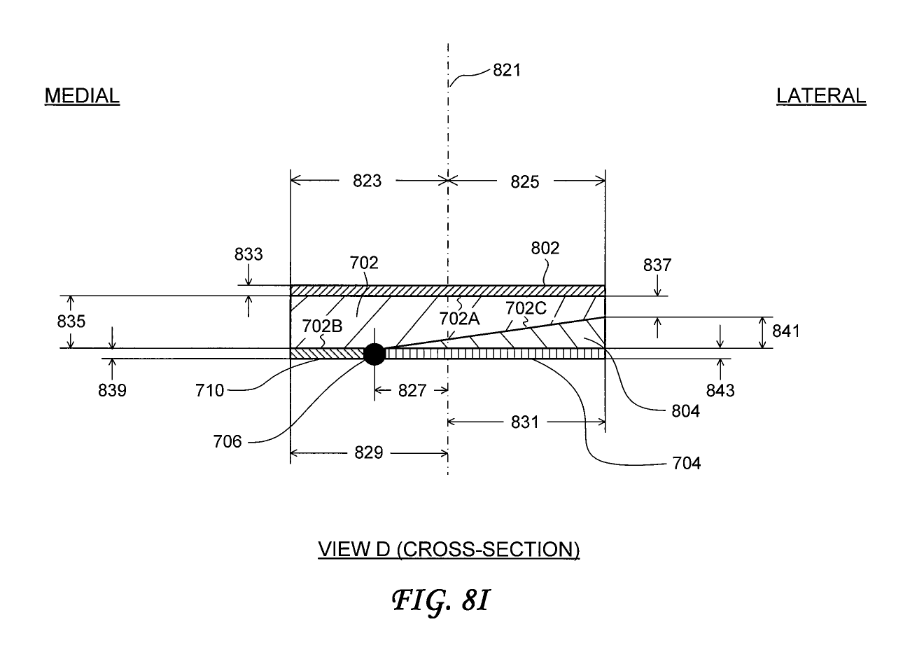

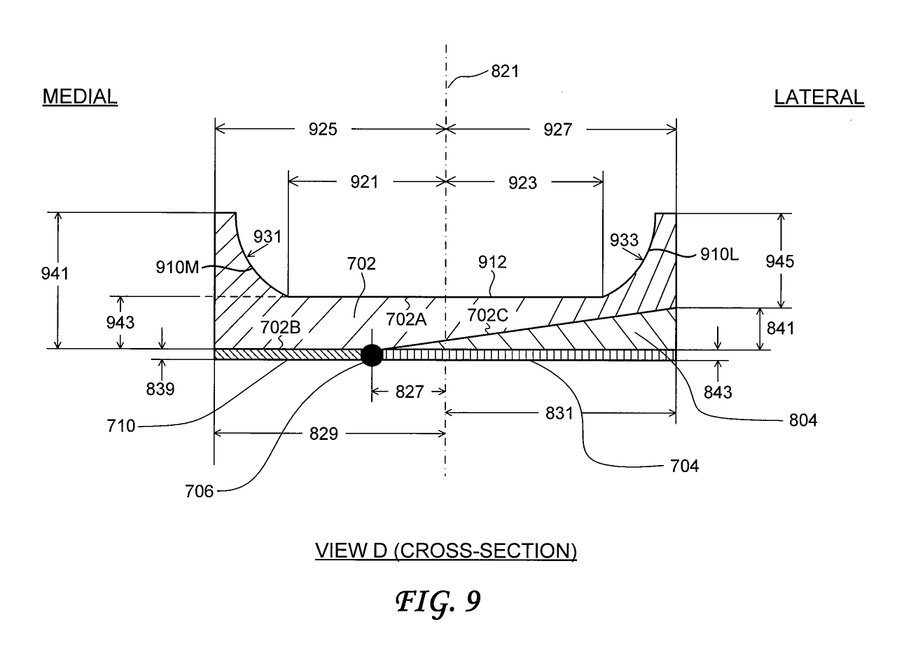

FIG. 9 (View D cross-section) shows a dimensional schematic of a rearfoot post similar to that shown in FIG. 8I, except that a heel cup is integrated into the platform 702 (in this instance, the heel cup is referred to as an integral heel cup). In other embodiments, a separate heel cup is attached to the platform 702. Note that the embodiment shown has no shell. Other embodiments have a shell. Dimensions in FIG. 9 corresponding to the dimensions in FIG. 8I are called out with the same reference numbers. The additional dimensions in FIG. 9 are described below. All the dimensions are user-specified design parameters. The heel cup includes the heel cup bottom 912, the heel cup medial side 910M, and the heel cup lateral side 910L. The heel cup bottom 912 is flat; the heel cup medial side 910M has a radius 931; and the heel cup lateral side 910L has a radius 933.

The distance between midline 821 and the outside edge of the heel cup on the medial side is distance 925. The distance between midline 821 and the outside edge of the heel cup on the lateral side is distance 927. The distance between midline 821 and the medial edge of heel cup bottom 912 is distance 921. The distance between midline 821 and the lateral edge of heel cup bottom 912 is distance 923.

The thickness of the platform 702 on the medial side is thickness 941. The thickness of the platform 702 on the lateral side is thickness 945. The thickness of the platform 702 at the medial edge of the heel cup bottom 912 is thickness 943.

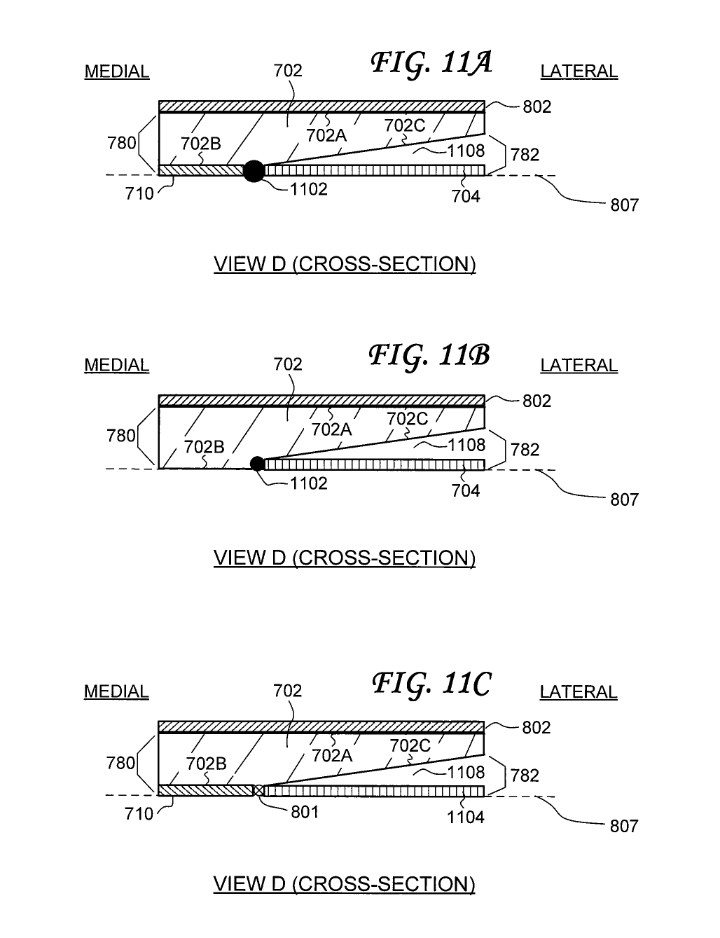

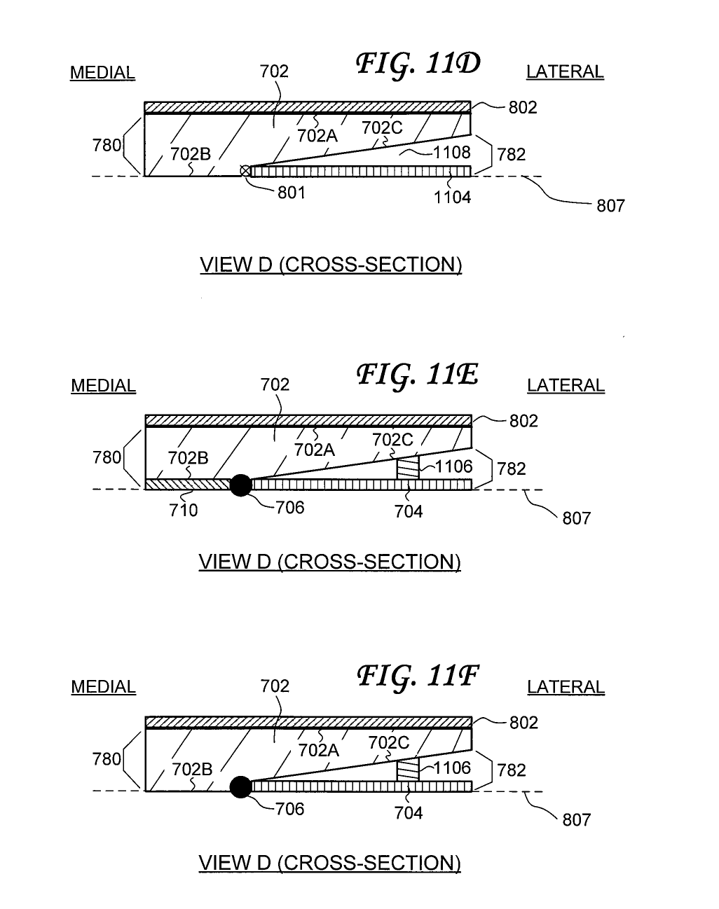

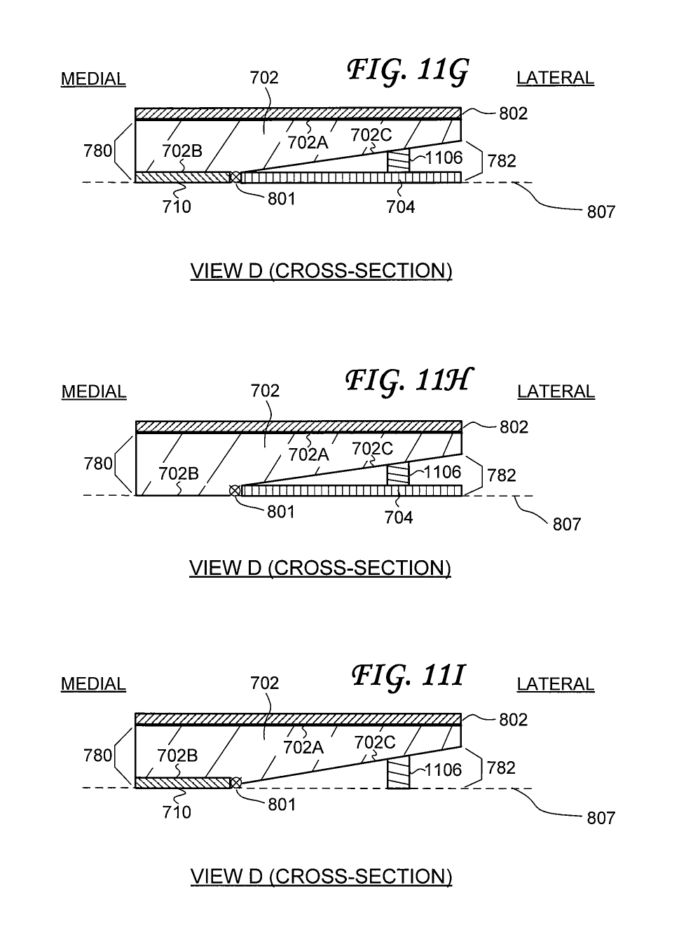

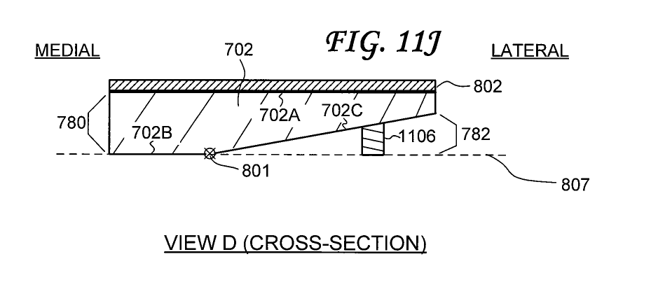

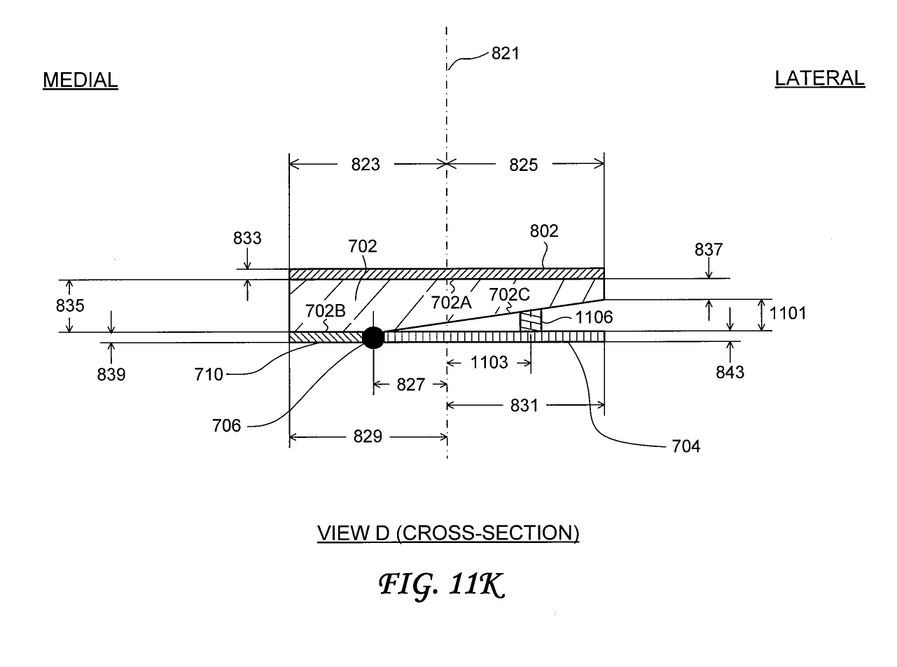

FIG. 11A-FIG. 11J (View D cross-section) show configurations of rearfoot posts according to embodiments of the invention in which the elastic segment includes a spring instead of an elastomer. Note that the embodiments shown include a shell. Other embodiments have no shell.

In the embodiment shown in FIG. 11A, the movable plate 704 is operatively coupled to the fixed plate 710 by a spring-loaded hinge 1102. Various spring-loaded hinges, including hinges with hidden coaxial springs, can be used. In this instance, compression-expansion zone 708 (see FIG. 7G) is an air gap 1108. The elastic segment 782 includes air gap 1108, movable plate 704, and spring-loaded hinge 1102.

The embodiment shown in FIG. 11B is similar to that shown in FIG. 11A, except that the fixed plate 710 is absent. The movable plate 704 is operatively coupled to the platform 702 by the spring-loaded hinge 1102.

The embodiment shown in FIG. 11C is similar to that shown in FIG. 11A, except that the spring-loaded hinge 1102 is absent. The movable plate is a spring plate 1104 that is attached to the fixed plate 710 along the axis of rotation (AOR) 801. In one embodiment, the fixed plate 710 and the spring plate 1104 is fabricated from a single sheet of material. Any material suitable for a spring, including various metals and plastics, can be used. The elastic segment 782 includes air gap 1108 and spring plate 1104.

The embodiment shown is FIG. 11D is similar to the embodiment shown in FIG. 11C, except that the fixed plate is absent. The spring plate 1104 is attached to the platform 702 along the axis of rotation 801.

The embodiment shown in FIG. 11E is similar to that shown in FIG. 8A, except that the elastomer is absent. The elastic segment 782 includes the movable plate 704 and a spring 1106 disposed between inclined surface 702C and the movable plate 704. Various types of springs, including coil springs and leaf springs, can be used. In some embodiments, a single spring is used; in other embodiments, two or more springs are used.

The embodiment shown in FIG. 11F is similar to that shown in FIG. 11E, except that the fixed plate is absent. The movable plate 704 is operatively coupled to the platform 702 by the hinge 706.

The embodiment shown in FIG. 11G is similar to that shown in FIG. 11E, except that the hinge is absent, and the movable plate 704 is attached to the fixed plate 710 along the axis of rotation 801. In one embodiment, the movable plate 704 and the fixed plate 710 are formed from a single sheet of material.

The embodiment shown in FIG. 11H is similar to the embodiment shown in FIG. 11G, except that the fixed plate is absent. The movable plate 704 is attached to the platform 702 along the axis of rotation 801.

The embodiment shown in FIG. 11I is similar to that shown in FIG. 11G, except that the movable plate is absent. The bottom of spring 1106 rests on reference plane 807. For example, reference 807 can represent the top surface of the insole of a shoe. This embodiment is advantageous if the insole is fabricated from a hard material and is advantageous for an orthotic integrated into prescription shoes. In some embodiments, a cover, cap, or plate can be attached to the bottom of the spring 1106 (in this instance, the plate is not attached to the fixed plate 710). In this instance, the elastic segment 782 includes spring 1106.

The embodiment shown in FIG. 11J is similar to that shown in FIG. 11I, except that the fixed plate is absent. In some embodiments, a cover, cap, or plate can be attached to the bottom of the spring 1106 (in this instance, the plate is not attached to the platform 702).

FIG. 11K shows a dimensional schematic of the rearfoot post previously shown in FIG. 11A. The dimensional schematic shown in FIG. 11K is similar to the one previously shown in FIG. 8I. Dimensions in FIG. 11K corresponding to the dimensions in FIG. 8I are called out with the same reference numbers. The additional dimensions shown in FIG. 11K are the following: distance 1103 is the distance between the midline 821 and the center axis of spring 1106, and spacing 1101 is the spacing (on the lateral edge) between the platform 702 and the movable plate 704 when the spring 1106 is in the uncompressed state. All the dimensions are user-specified design parameters.

In the embodiments shown in FIG. 11E-FIG. 11J, the elastic segment includes one or more springs (a mixture of different types of springs can be used). In other embodiments, a piston can be used instead of a spring. Various pistons can be used: for example, a spring-loaded piston or a fluid-filled piston. Fluid-filled pistons include pneumatic pistons (filled with air or another gas) and hydraulic pistons (filled with a liquid or gel). A mixture of various springs and pistons can be used.

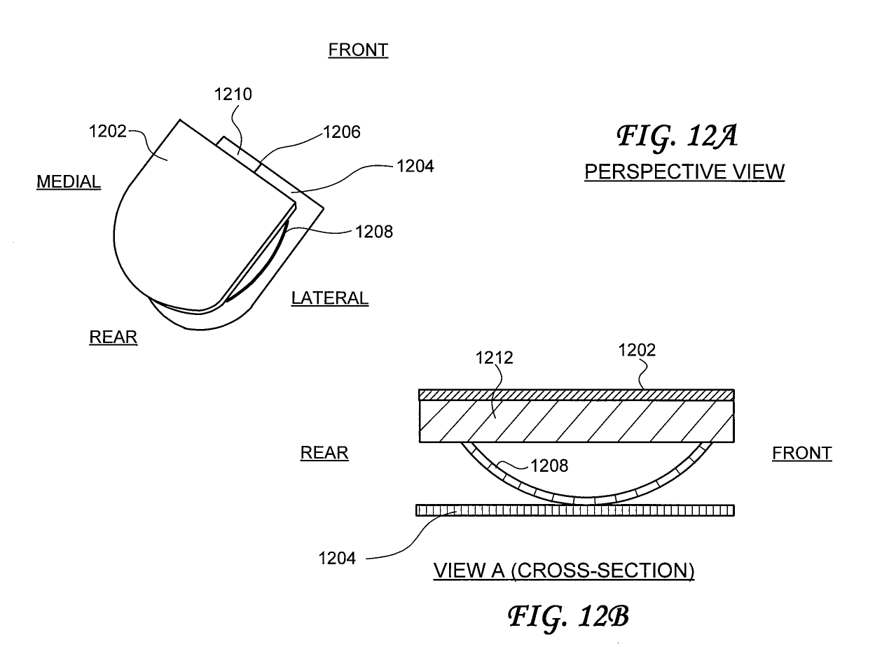

FIG. 12A (perspective view) and FIG. 12B (View A cross-section) show an embodiment of a rearfoot post with a U-shaped shell 1202. Platform 1212 (not visible in FIG. 12A) is attached to the bottom of shell 1202. Fixed plate 1210 is attached to the bottom medial surface of platform 1212, and movable plate 1204 is operatively coupled to fixed plate 1210 by hinge 1206. Spring 1208 is disposed between the platform 1212 and the movable plate 1204. In the example shown, spring 1208 is a leaf spring.

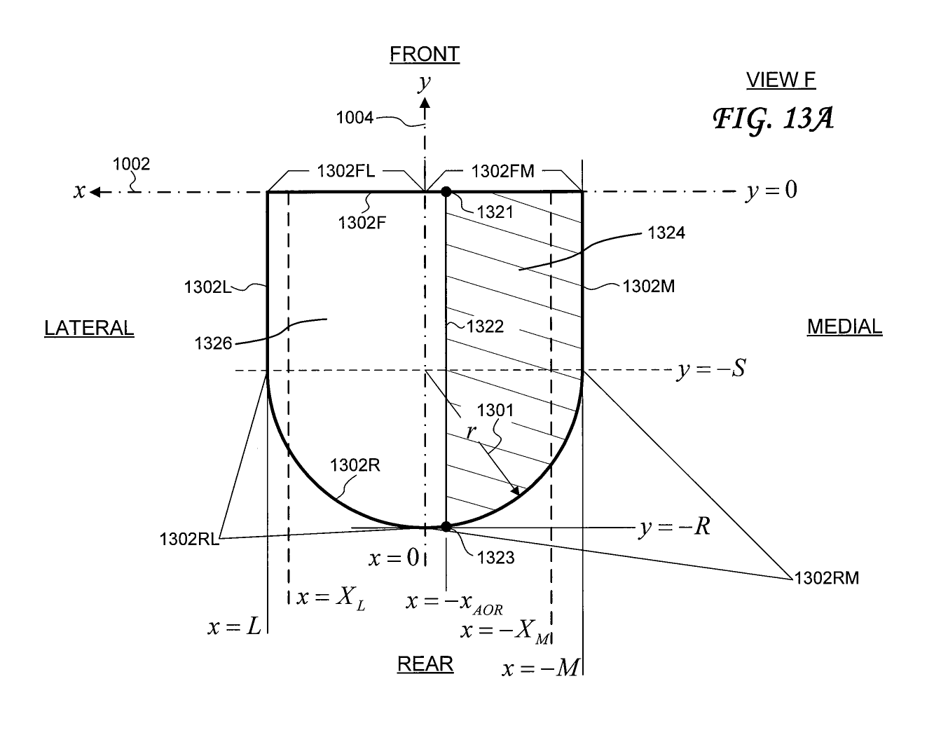

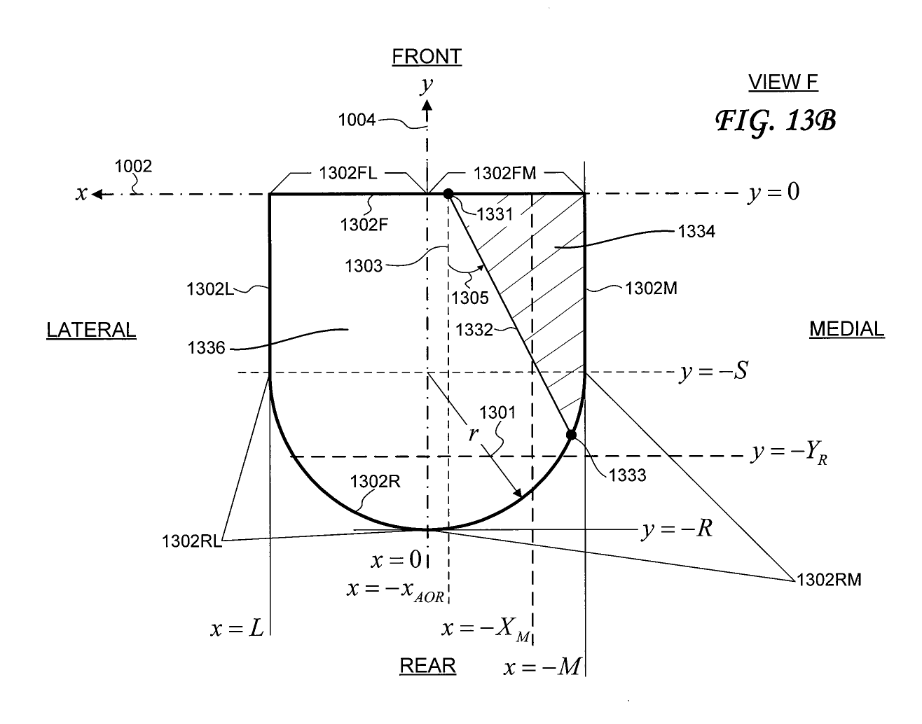

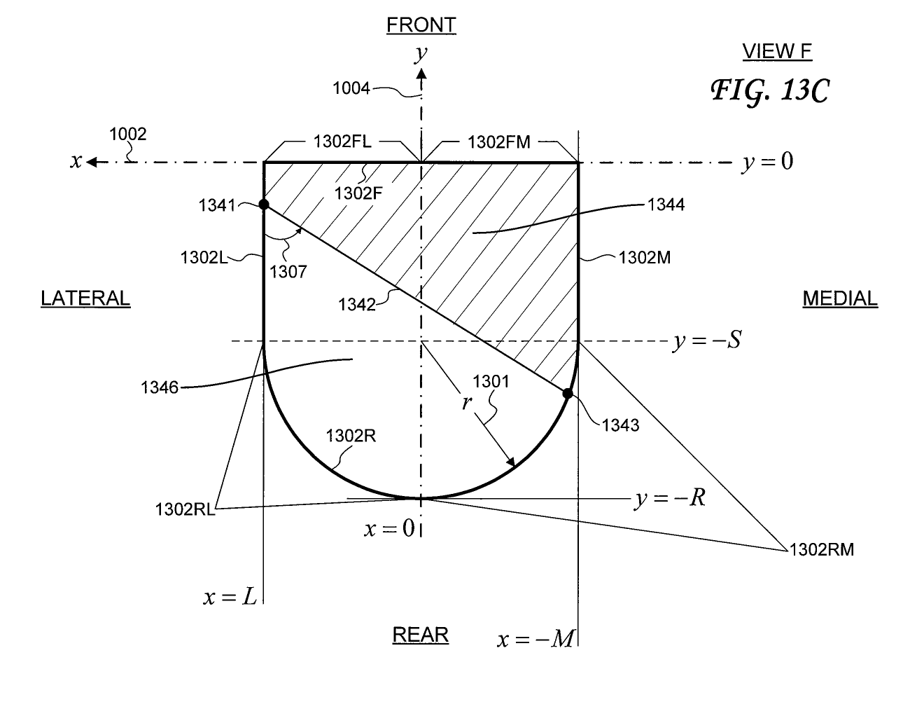

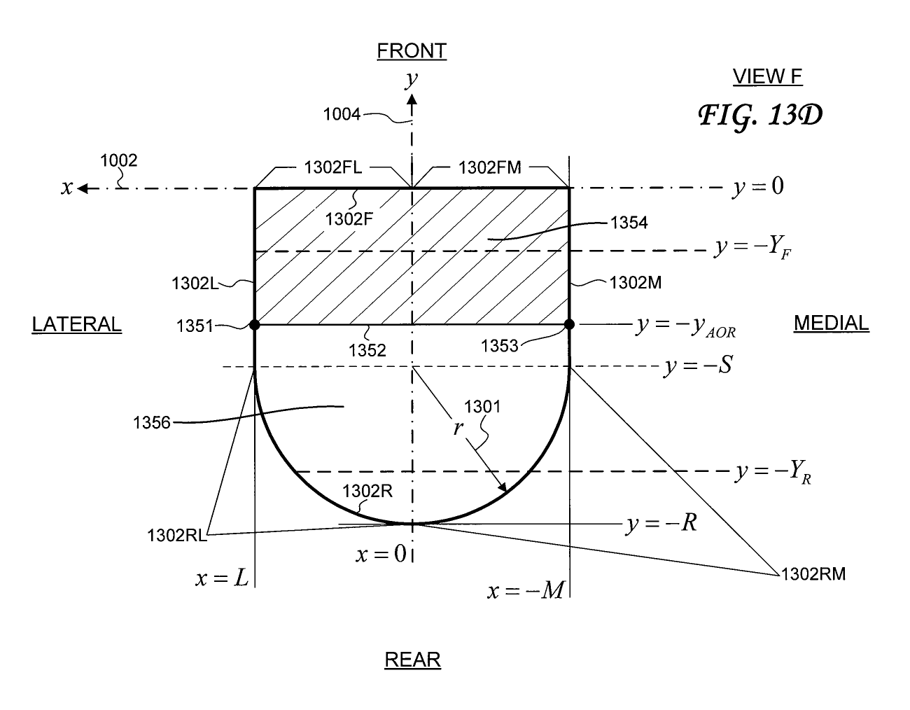

The location and orientation of the axis of rotation of the rearfoot post can be user-specified to treat specific foot conditions. The axis of rotation of the rearfoot post lies within the rearfoot post. In general, the stop segment and the elastic segment are operatively coupled along the axis of rotation of the rearfoot post such that the elastic segment can rotate with respect to the stop segment about the axis of rotation of the rearfoot post. The combination of a stop segment and an elastic segment limits the range of rotation: it stops rotation from occurring over a first user-specified range and allows rotation to occur over a second user-specified range. If the rearfoot post does not include a hinge, then the axis of rotation of the rearfoot post coincides with the boundary between the stop segment and the elastic segment. If the rearfoot post includes a hinge, then the axis of rotation of the rearfoot post coincides with the axis of rotation of the hinge (also referred to as the axis of the hinge). The axis of rotation of the rearfoot post can be specified by two end points along the periphery of the bottom surface of the rearfoot post. FIG. 13A-FIG. 13D (View F) show four configurations of an axis of rotation.

Refer to FIG. 13A. The bottom surface of the rearfoot post has a periphery including front edge 1302F, lateral edge 1302L, medial edge 1302M, and rear edge 1302R. The x-axis 1002 is placed along the front edge 1302F of the rearfoot post. The y-axis 1004 is placed along the midline of the rearfoot post. The front edge 1302F is defined by the line segment with end points at (x, y)₁=(L, 0) and (x, y)₂=(−M, 0). The lateral edge 1302L is defined by the line segment with end points at (x, y)₁=(L, 0) and (x, y)₂=(L, −S). The medial edge 1302M is defined by the line segment with end points at (x, y)₁=(−M, 0) and (x, y)₂=(−M, −S). The rear edge 1302R is defined by the semicircular arc with end points at (x, y)₁=(L, −S) and (x, y)₂=(−M, −S) and a radius 1311 of r=(L+M)/2.

The region of the bottom surface of the rearfoot post between the midline and the lateral edge (L≧x≧0) is referred to as the lateral region. The region of the bottom surface of the rearfoot post between the midline and the medial edge (−M≦x≦0) is referred to as the medial region. The periphery can be further partitioned into a lateral periphery and a medial periphery. The lateral periphery is defined by the locus of points on the periphery such that x≧0. The medial periphery is defined by the locus of points on the periphery such that x≦0.

In the example shown in FIG. 13A, the front edge 1302F is partitioned into the lateral front edge 1302FL and the medial front edge 1302M. The rear edge 1302R is partitioned into the lateral rear edge 1302RL and the medial rear edge 1302RM. The lateral periphery is the union of the lateral front edge 1302FL, the lateral edge 1302L and the lateral rear edge 1302RL. The medial periphery is the union of the medial front edge 1302FM, the medial edge 1302M, and the medial rear edge 1302RM.

In FIG. 13A, a representative axis of rotation (AOR) 1322 is shown. AOR 1322 is parallel to the y-axis and is defined by the two end points (endpoint-1 1321, endpoint-2 1323). Endpoint-1 1321 is located on the front edge 1302F of the rearfoot post. The coordinates of endpoint-1 1321 are (x, y)₁=(x₁, y₁)=(−x_{AOR}, 0). In general, the value of x₁ falls within the range L>X_{L}≧x₁≧−x_{M}>−M, where X_{L} is a user-specified design limit towards the lateral edge and −X_{M} is a user-specified design limit towards the medial edge. In an advantageous embodiment for control of subtalar pronation, the axis of rotation is located in the medial region, 0≧x₁≧−X_{M}. AOR 1322 partitions the rearfoot post into the stop segment 1324 and the elastic segment 1326. For illustration purposes, stop segment 1324 (−x_{AOR}≧x≧−M) is shown as a shaded region.

FIG. 13B shows an embodiment in which the axis of rotation is oriented at an offset angle. A representative axis of rotation 1332 is shown. AOR 1332 is defined by the two end points (endpoint-1 1331, endpoint-2 1333). Endpoint-1 1331 is located on the front edge 1302F of the rearfoot post. The coordinates of endpoint-1 1331 are (x, y)₁=(x₁, y₁)=(x_{AOR}, 0). In general, the value of x₁ falls within the range L>X_{L}≧x₁≧−X_{M}>−M, where X_{L} is a user-specified design limit towards the lateral side and −X_{M} is a user-specified design limit towards the medial side. AOR 1332 is offset by the offset angle φ=φ_{AOR} 1305 with respect to a reference axis 1303 that is parallel to the y-axis and intersects endpoint-1 1331. In general, the offset angle φ falls within the range of ±90°, where the positive direction is counter-clockwise as shown. In an advantageous embodiment for control of subtalar pronation, endpoint-1 1331 is located on the front medial edge 1302FM (0≧x₁≧−X_{M}), and the offset angle φ is positive (0<φ≦Φ<90°), where Φ is a user-specified maximum offset angle (note, in general, Φ is a function of x₁). AOR 1332 partitions the rearfoot post into the stop segment 1334 and the elastic segment 1336. For illustration purposes, stop segment 1334 is shown as a shaded region.

FIG. 13C shows an embodiment in which the axis of rotation AOR 1342 is defined by the two end points (endpoint-1 1341, endpoint-2 1343). Endpoint-1 1341 is located on the lateral edge 1302L of the rearfoot post. The coordinates of endpoint-1 1341 are (x, y)₁=(x₁, y₁)=(L, −y_{AOR}). The offset angle φ 1307 is positive (0<φ≦Φ<90°), where Φ is a user-specified maximum offset angle (note, in general, Φ is a function of −y_{AOR}). AOR 1342 partitions the rearfoot post into the stop segment 1344 and the elastic segment 1346. For illustration purposes, stop segment 1344 is shown as a shaded region.

In general, the endpoint-1 1341 can also fall on the rear lateral edge 1302RL. In general, the value of y₁ falls within the range 0>−Y_{F}≧y₁≧−Y_{R}>−R, where −Y_{F} is a user-specified design limit towards the front edge and −Y_{R} is a user-specified design limit towards the rear edge.

FIG. 13D shows an embodiment in which the axis of rotation is parallel to the x-axis. A representative axis of rotation 1352 is shown. AOR 1352 is defined by the two end points (endpoint-1 1351, endpoint-2 1353). Endpoint-1 1351 is located on the lateral edge 1302L of the rearfoot post. Endpoint-2 1353 is located on the medial edge 1302M of the rearfoot post. The coordinates of endpoint-1 1351 are (x, y)₁=(x₁, y₁)=(L, −y_{AOR}). The coordinates of endpoint-2 1353 are (x, y)₂=(x₂, y₂)=(−M, −y_{AOR}).

In general, the end points can also fall on the rear edge 1302R. In general, the value of y₁=y₂ falls within the range 0>−Y_{F}≧y₁≧−Y_{R}>−R, where −Y_{F} is a user-specified design limit towards the front edge and −Y_{R} is a user-specified design limit towards the rear edge.

AOR 1352 partitions the rearfoot post into the stop segment 1354 and the elastic segment 1356. For illustration purposes, stop segment 1354 (0≧y≧−y_{AOR}) is shown as a shaded region.

As discussed above, a rearfoot post can be used with an orthotic that is configured to extend along the bottom surface of the foot. An orthotic can be configured to extend along a portion of or the entirety of the bottom surface of the foot. Herein, the body of an orthotic refers to the portion of the orthotic not including the rearfoot post itself. The portion of the body of the orthotic configured to extend along the bottom surface of the foot in front of the heel is referred to as the front portion of the body of the orthotic (the front portion of the body of the orthotic can be configured to extend along a portion of or the entirety of the front portion of the bottom surface of the foot). The portion of the body of the orthotic configured to extend along the bottom surface of the heel is referred to as the heel portion of the body of the orthotic (the heel portion of the body of the orthotic can be configured to extend along a portion of or the entirety of the heel of the bottom surface of the foot).

The body of the orthotic can be configured to have only a front portion. In some embodiments, the body of the orthotic and the rearfoot post are separate units. In some embodiments, the body of the orthotic is attached to the rearfoot post.

The body of the orthotic can be configured to have a heel portion and a front portion. In some embodiments, the rearfoot post is attached to the bottom of the heel portion of the body of the orthotic.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A rearfoot post comprising:
a stop segment; and
an elastic segment operatively coupled to the stop segment along an axis of rotation of the rearfoot post.

2. The rearfoot post of claim 1, further comprising:
a hinge operatively coupled to the stop segment and to the elastic segment, wherein the axis of rotation of the rearfoot post comprises an axis of rotation of the hinge.

3. The rearfoot post of claim 1, wherein the stop segment comprises a material with a hardness of greater than or equal to approximately 35 Shore A durometer.

4. The rearfoot post of claim 1, wherein the elastic segment comprises a spring.

5. The rearfoot post of claim 1, wherein the elastic segment comprises an elastomer.

6. The rearfoot post of claim 5, wherein the elastomer comprises a material with a rebound resilience of approximately 12-25%.

7. The rearfoot post of claim 6, further comprising:
a plate attached to a bottom surface of the elastomer.

8. The rearfoot post of claim 1, wherein the elastic segment comprises a fluid-filled bladder.

9. The rearfoot post of claim 1, wherein the elastic segment comprises a spring-loaded piston.

10. The rearfoot post of claim 1, wherein the elastic segment comprises a fluid-filled piston.

11. The rearfoot post of claim 1, further comprising:
a heel cup.

12. The rearfoot post of claim 11, wherein the heel cup comprises:
a curved bottom;
a curved medial side wall; and
a curved lateral side wall.

13. The rearfoot post of claim 11, wherein the heel cup comprises:
a flat bottom;
a curved medial side wall; and
a curved lateral side wall.

14. The rearfoot post of claim 11, further comprising:
a shell attached to the heel cup.

15. The rearfoot post of claim 1, wherein the stop segment comprises a platform comprising:
a top surface;
a bottom surface; and
an inclined surface.

16. The rearfoot post of claim 15, further comprising:
a plate attached to the bottom surface of the platform.

17. The rearfoot post of claim 15, further comprising:
a shell attached to the top surface of the platform.

18. The rearfoot post of claim 15, wherein the top surface of the platform is curved.

19. The rearfoot post of claim 15, wherein the top surface of the platform is flat.

20. The rearfoot post of claim 15, further comprising:
an elastomer disposed below the inclined surface of the platform.

21. The rearfoot post of claim 20, further comprising:
a plate attached to a bottom surface of the elastomer.

22. The rearfoot post of claim 15, further comprising:
an elastomer disposed below the inclined surface of the platform;
a plate attached to a bottom surface of the elastomer; and
a hinge operatively coupled to the platform and to the plate.

23. The rearfoot post of claim 15, further comprising:
an elastomer disposed below the inclined surface of the platform; and
a plate attached to the bottom surface of the platform and to a bottom surface of the elastomer.

24. The rearfoot post of claim 15, further comprising:
an elastomer disposed below the inclined surface of the platform;
a first plate attached to the bottom surface of the platform; and
a second plate attached to a bottom surface of the elastomer.

25. The rearfoot post of claim 24, wherein the first plate and the second plate are operatively coupled.

26. The rearfoot post of claim 15, further comprising:
an elastomer disposed below the inclined surface of the platform;
a first plate attached to the bottom surface of the platform;
a second plate attached to a bottom surface of the elastomer; and
a hinge operatively coupled to the first plate and to the second plate.

27. The rearfoot post of claim 15, further comprising:
a spring disposed below the inclined surface.

28. The rearfoot post of claim 27, wherein the spring comprises a spring plate operatively coupled to the platform.

29. The rearfoot post of claim 15, further comprising:
a plate operatively coupled to the platform and disposed below the inclined surface of the platform; and
a spring disposed between the plate and the inclined surface of the platform.

30. The rearfoot post of claim 15, further comprising:
a first plate attached to the bottom surface of the platform;
a second plate disposed below the inclined surface of the platform;
a hinge operatively coupled to the first plate and to the second plate; and
a spring disposed between the inclined surface of the platform and the second plate.

31. The rearfoot post of claim 15, further comprising:
a plate disposed below the inclined surface of the platform; and
a spring-loaded hinge operative coupled to the platform and to the plate.

32. The rearfoot post of claim 15, further comprising:
a first plate attached to the bottom surface of the platform;
a second plate disposed below the inclined surface of the platform; and
a spring-loaded hinge operatively coupled to the first plate and to the second plate.

33. An orthotic comprising:
a body; and
a rearfoot post comprising:
a stop segment; and
an elastic segment operatively coupled to the stop segment along an axis of rotation of the rearfoot post.

34. The orthotic of claim 33, wherein the body is configured to extend only along at least a portion of the bottom surface of a foot in front of the heel of the foot.

35. The orthotic of claim 34, wherein the body is attached to the rearfoot post.

36. The orthotic of claim 34, wherein the body is not attached to the rearfoot post.

37. The orthotic of claim 33, wherein:
the body is configured to extend along at least a portion of the bottom surface of a foot in front of the heel of the foot and along at least a portion of the bottom surface of the foot within the heel of the foot; and
the rearfoot post is attached to the body.
